(19) United States
(12) Patent Application Publication
(10) Pub. No.: US 2010/0099958 A1
(43) Pub. Date: Apr. 22, 2010

(54) Method of identifying when a patient undergoing hemodialysis is at increased risk of death

(75) Inventors: Peter Kotanko, New York, NY (US); Stephan Thijssen, New York, NY (US); Len Usvyat, Philadelphia, PA (US); Nathan W. Levin, New York, NY (US)

Correspondence Address: Hamilton, Brook, Smith & Reynolds, P.C., 530 Virginia Road, P.O.Box 9133, Concord, MA, 01742, US

(73) Assignee: Fresenius Medical Care Holdings Inc., Waltham, MA (US)

(21) Appl. No.: 12/587,941

(22) Filed: Oct. 15, 2009

Related U.S. Application Data
(60) Provisional application No. 61/196,255, filed on Oct. 16, 2008.

(51) Int. Cl. A61B 5/00

(52) U.S. Cl. 600/301

(57) ABSTRACT

The invention is directed to a method of identifying a patient undergoing periodic hemodialysis treatments at increased risk for death that includes determining at least one of the patient's systolic blood pressure, serum albumin level, body weight, and body temperature at periodic hemodialysis treatments, and identifying a patient as having an increased risk for death if the patient has a substantial change in the rate of decline of at least one of the patient's systolic blood pressure, serum albumin level, body weight, and body temperature. The invention is also directed to a method of identifying an increased mortality risk factor for a patient undergoing periodic hemodialysis treatment. The method includes analyzing data in deceased patients that were previously undergoing periodic hemodialysis treatments by performing a longitudinal analysis backwards in time of changes in a clinical or biochemical parameter the patients, and identifying a substantial change in the rate of decline or the rate of increase in a clinical or biochemical parameter before death of the patients.

### RELATED APPLICATION

This application claims the benefit of U.S. Provisional Application No. 61/196,255, filed on Oct. 16, 2008.

The entire teachings of the above application are incorporated herein by reference.

### BACKGROUND OF THE INVENTION

Despite significant advances in hemodialysis (HD) technology, the mortality risk of chronic HD patients remains well above that seen in the general population, where individuals younger than 30 years of age have an on average a 4-times longer age-adjusted live expectancy than HD patients of comparable age, and HD patients age 65 or older have a mortality risk 6 times higher than the general population. Cardiovascular disease and infectious disease are among the leading causes of mortality, and the difference in mortality risk between HD patients and the general population is most pronounced for heart disease with three fold higher death rates (180.8 versus 49.8 deaths per 1,000 patient years) in individuals age 45 to 64. See United States Renal Data System, Mortality and causes of death, Annual Data Report (2007).

Current epidemiologic studies seeking to investigate the determinants of mortality risk in dialysis patients usually consider either cross-sectional baseline characteristics (e.g., mean systolic blood pressure in the first 3 months after start of dialysis; serum albumin levels after 6 months) or time-dependent analyses, most commonly time-dependent Cox regression models. Patients are frequently stratified into groups based on descriptive characteristics such as tertiles. Of note, in many of these studies, the first date of dialysis is taken as the reference point.

Despite such improvements in hemodialysis technology and patient tracking, chronic hemodialysis patients continue to experience an inordinately high mortality rate. Therefore, there is a need for an improved method of identifying hemodialysis patients at increased risk of death, in order to trigger earlier diagnostic and therapeutic interventions and consequently reduce patient mortality.

### SUMMARY OF THE INVENTION

The present invention is directed to a method of identifying a patient undergoing periodic hemodialysis treatments at increased risk for death. The method includes determining at least one of the patient's systolic blood pressure, serum albumin level, body weight and body temperature periodically while the patient is undergoing hemodialysis treatments, and identifying a patient as having an increased risk for death if the patient has a substantial change in the rate of decline of at least one of the patient's systolic blood pressure, serum albumin level, body weight, and body temperature. In a preferred embodiment, a determination that the patient's systolic blood pressure, serum albumin level, body weight, and body temperature all have had a substantial change in the rate of decline is employed to identify patients at increased risk of death. In another preferred embodiment, identifying the patient as having an increased risk of death is accomplished within a sufficient lead time to allow for a therapeutic intervention to decrease the patient's risk of death.

The present invention is also directed to a method of identifying an increased mortality risk factor for a patient undergoing periodic hemodialysis treatment. The method includes analyzing data in deceased patients that were previously undergoing periodic hemodialysis treatments by performing a longitudinal analysis backwards in time of changes in a clinical or biochemical parameter the patients, and identifying a substantial change in the rate of decline or the rate of increase of a clinical or biochemical parameter before death of the patients.

The methods of this invention enable physicians and/or other health-care professionals to initiate timely diagnostic and therapeutic interventions to hemodialysis patients at increased risk of death and thereby reduce mortality of such patients.

### BRIEF DESCRIPTION OF THE DRAWINGS

The foregoing will be apparent from the following more particular description of example embodiments of the invention, as illustrated in the accompanying drawings. The drawings are not necessarily to scale, emphasis instead being placed upon illustrating embodiments of the present invention.

FIG. 1 is a graph of linear splines of post-dialysis body weight of hemodialysis patients as a function of time before death; knot point at 12 weeks before death.

FIG. 2 is a graph of linear splines of serum albumin concentration levels of hemodialysis patients as a function of time before death; knot point at 3 months before death.

FIG. 3 is a graph of linear splines of systolic blood pressure of hemodialysis patients as a function of time before death; knot point at 12 weeks before death.

FIG. 4 is a graph of linear splines of body temperature of hemodialysis patients as a function of time before death; knot point at 12 weeks before death.

### DETAILED DESCRIPTION OF THE INVENTION

The present invention is directed to a method of identifying a patient at increased risk for death when the patient is undergoing periodic hemodialysis treatments. The method includes determining at least one of the patient's systolic blood pressure, serum albumin level, body weight, and body temperature at periodic hemodialysis treatments. The patient is identified as having an increased risk for death if the patient has a substantial increase in the rate of decline of at least one of the patient's systolic blood pressure, serum albumin level, body weight, and body temperature. The measurement of at least one of these clinical parameters includes the measurement of any combination of the patient's systolic blood pressure, serum albumin level, body weight, and body temperature. In a preferred embodiment, a determination that the patient's systolic blood pressure, serum albumin level, body weight, and body temperature all have had a substantial increase in the rate of decline is employed to identify patients at increased risk of death.

The method is applied to a patient that is undergoing periodic hemodialysis treatments. Typically, periodic hemodialysis treatments are performed several days apart, for example, three times per week. The time period between treatments is not necessarily constant, however, because, for example, the patient can receive treatment after a shorter time period since the last treatment if the patient needs to shed excess fluid. The time period between treatments can be longer because of, for example, missed treatments or an illness acquired since the last treatment.

The methods of this invention apply to human patients that are undergoing hemodialysis treatment. The hemodialysis treatment of the patient is a treatment that replaces or supplements the normal function of the kidneys of a patient, due to the patient having a disease or condition that affects kidney function such as, for example, renal insufficiency, renal failure, or kidney disease.

The measurements of the patient's systolic blood pressure, serum albumin level, body weight, and body temperature are taken using methods well known in the art. The measurements of the aforementioned clinical or biochemical parameters can be performed either before or after each hemodialysis treatment, or both, or only performed after a certain time period, or at every certain number of treatments, or at irregular intervals. For example, the measurement of systolic blood pressure is usually taken before each treatment, but can also be taken after each treatment, or both before and after each treatment. The measurement of albumin levels is usually taken once a month, but can also be taken more often. The measurement of body weight is usually taken before each treatment, but can also be taken after each treatment. The measurement of body temperature is preferentially taken before each treatment, but can also be taken after each treatment. Of course, the measurements of the patient's clinical and biochemical parameters could also be taken in between hemodialysis treatments.

The importance of determining a substantial increase in the rate of decline of the patient's systolic blood pressure, serum albumin level, body weight, and body temperature was uncovered by focusing specifically on the time-course of these clinical parameters before death in a large sample of hemodialysis patients. In this analysis, the reference point for the analysis was the patient's date of death, and the analysis looked back in time from that point, in order to uncover what changes in clinical parameters preceded demise. This retrospective record review included a data set of 2,462 in-center maintenance HD patients who expired between Jul. 1, 2005 and Apr. 30, 2008. Patients' monthly serum albumin levels were extracted for the 24 months preceding the date of death. Similarly, the median weekly post-dialysis weight was extracted for the 104 weeks prior to death. Causes of death (COD), recorded using ICD-9 codes, were retrieved from patient record sheets. See The International Classification of Diseases, 9^{th} Revision, Clinical Modification, (ICD-9-CM), National Center for Health Statistics and Centers for Medicare & Medicaid Services (2007). Three broad COD categories (cardiovascular, cerebrovascular, and infectious) were included in the analyses. Going back in time allowed an analysis of events occurring in the days, weeks, and months prior to demise. This is, in principle, a longitudinal data analysis backwards in time with death as the common end point. The defining feature of such a longitudinal analysis is that measurements of the same individual are taken repeatedly over time, thereby allowing the direct study of change over time. Measurement variability stems from three sources, between-subject heterogeneity, within-subject variability, and (random) measurement errors. With repeated measurements available the individual patients' changes in responses over time can be studied. In addition, the mean response of a group (for example, gender; race; co-morbidities) can be modeled.

The longitudinal analysis of patient albumin, systolic blood pressure, body weight, and body temperature was conducted using linear mixed effects models (LMMs). LMMs form a broad class of models which handle longitudinal data in a very general setting (e.g., the data can be unbalanced and mistimed). See G. M. Fitzmaurice, N. M. Laird, and J. H. Ware, Applied Longitudinal Analysis, (2004). In the LMMs employed, individual patient effects can be separated from population effects by treating the individual effects as random, while the population effects are regarded as fixed; the full model combines the random and the fixed effects. A powerful result is that subject response trajectories can be estimated in addition to the population response trajectory. In this application, a random intercept model was used. In this model, each subject has a distinct level of response which persists over time. The patient serves as his or her own control insofar as the dynamics between observed in two time periods are compared. To determine which random effects should be included in the models, the Bayesian Information Criterion (BIC) was used; this measure rewards a model with higher explanatory power, while penalizing for the inclusion of additional parameters. In this data analysis, the data were fit by linear spline functions, because these simple parametric curves can provide a parsimonious description of longitudinal trends. See D. Ruppert, M. P. Wand, and R. J. Carroll, Semiparametric Regression, (2003). Linear spline functions with a knot at 12 weeks before death were employed for systolic blood pressure, body weight, and body temperature. A knot point is the point in time where two spline functions intersect. Clearly, the choice of the location for the knot point is important with this kind of analysis. The knot point (12 weeks before death) was chosen by separating the data into two sets for processing, one data set including all the data up to 12 weeks before death, and the other data set including the data from 12 weeks before death to the patient's demise. The knot point (12 weeks before death) was chosen for the following reasons, (a) based on pilot descriptive data analysis which revealed an accelerated deterioration of body weight in the 12 weeks preceding death, and (b) because it was deemed that a lead time of 12 weeks was probably sufficient in many patients to intervene.

The time point chosen as the knot point generally depends on the clinical or biochemical parameter being analyzed, to provide sufficient time for an effective diagnostic or therapeutic patient intervention.

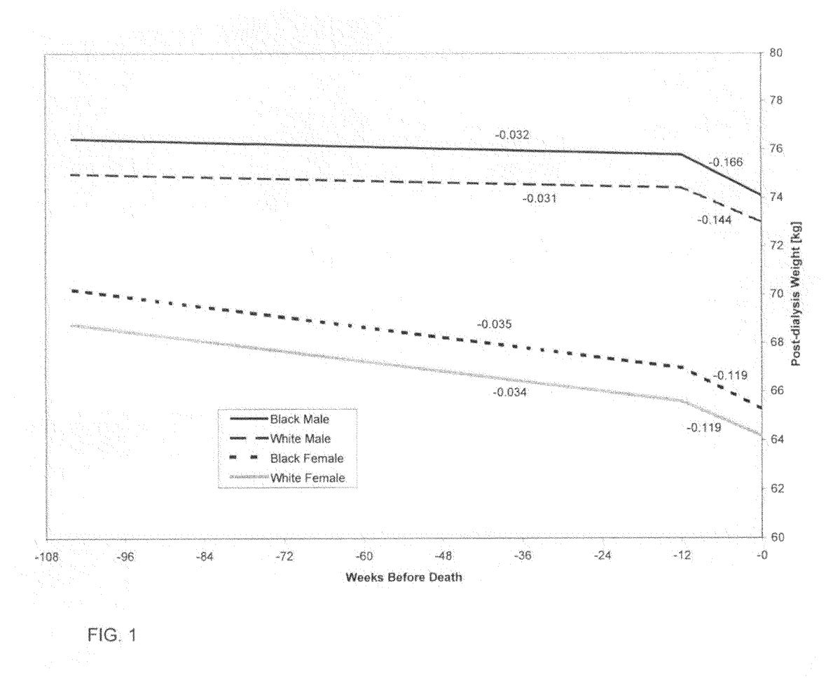

Turning now to FIG. 1, the results for post-dialysis body weight are shown for the data set. Four groups of dialysis patients, black and white males and females, all showed an increase in the rate of decline of post-dialysis body weight in the final 12 weeks of life, from about 0.03 kg/week to over about 0.1 kg/week. Therefore, in this study, for post-dialysis body weight, the rate of decline increased by a factor of about 3 in the final 12 weeks of life.

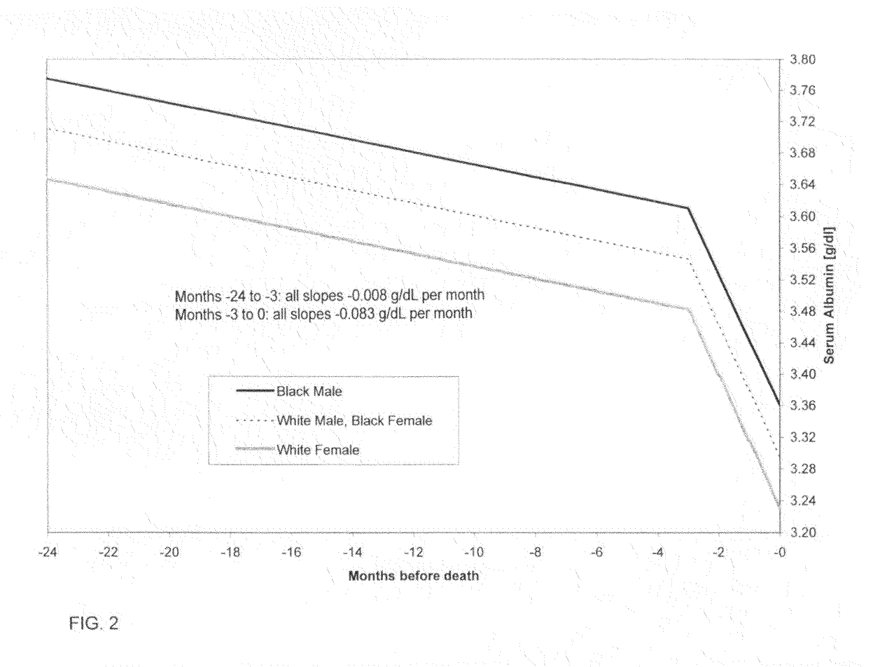

Turning now to FIG. 2, the results for serum albumin levels are shown for the data set. The knot point for the serum albumin data set was chosen at 3 months because the patient's serum albumin levels were measured at one month intervals. The four groups of dialysis patients showed an increase in the rate of decline of serum albumin levels in the final 3 months of life, from about 0.008 g/dL/month to over about 0.08 g/dL/month. Therefore, in this study, for serum albumin levels, the rate of decline increased by a factor of about 10 in the final 3 months of life.

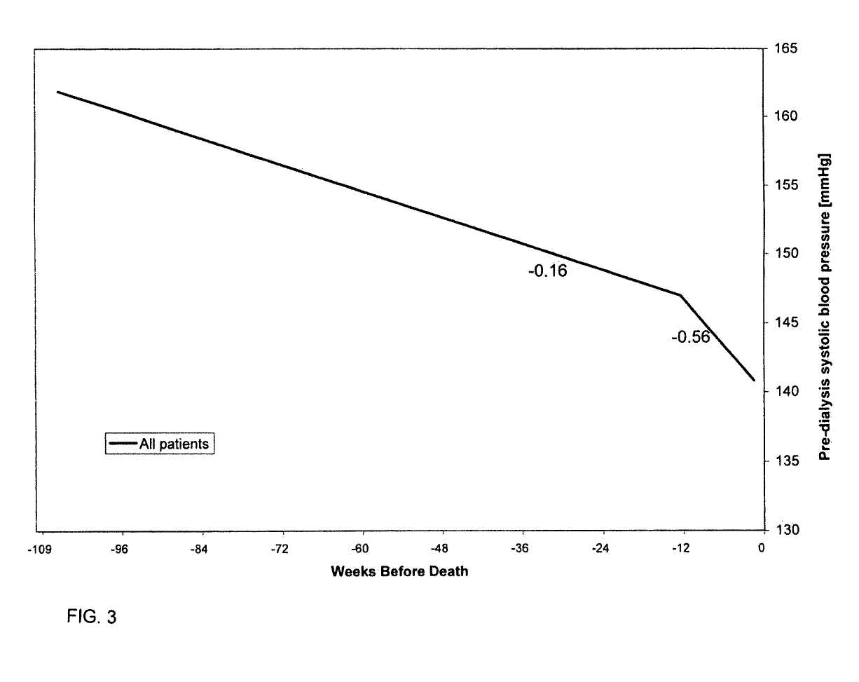

Turning now to FIG. 3, in a separate study of 1,799 hemodialysis patients, it was found that the average pre-dialysis systolic blood pressure of patients showed an increase in the rate of decline in the final 12 weeks of life, from about 0.16 mmHg/week to about 0.56 mmHg/week. Therefore, in this study, for pre-dialysis systolic blood pressure, the rate of decline increased by a factor of about 3 in the final 12 weeks of life.

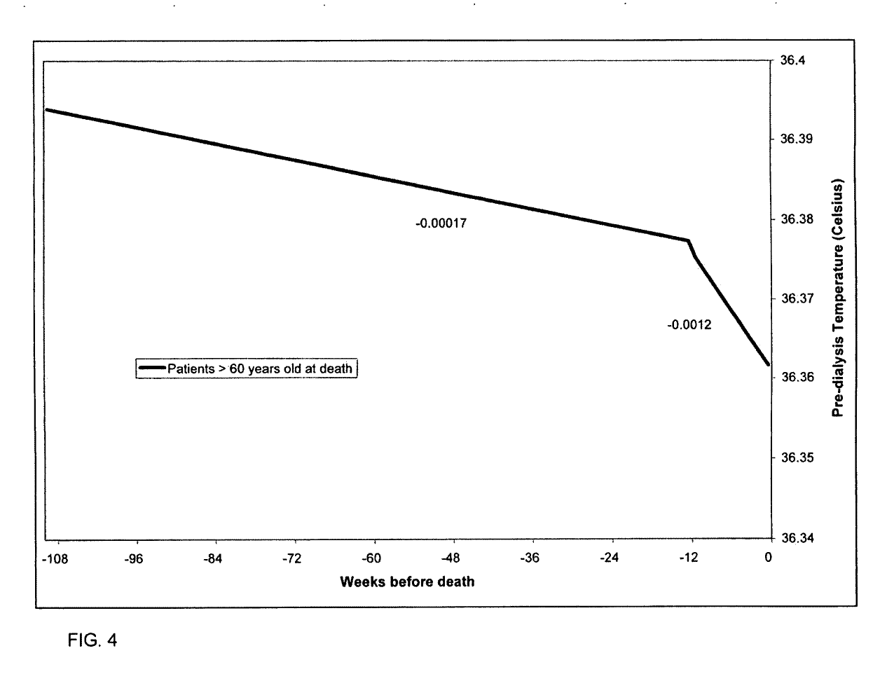

Turning now of FIG. 4, in another study of hemodialysis patients over 60 years old at death, it was found that the pre-dialysis body temperature of patients showed an increase in the rate of decline in the final 12 weeks of life, from about 0.00017 C/week to about 0.0012 C/week. Therefore, in this study, for body temperature, the rate of decline increased by a factor of about 7 in the final 12 weeks of life.

There are a number of other clinical or biochemical parameters that can be used to identify a hemodialysis patient at increased risk of death. Generally, these parameters can be grouped into four domains, the cardiovascular, nutrition, inflammatory, and anthropometric domains. Examples in the cardiovascular domain include the diastolic and mean blood pressure, and the pulse pressure and heart rate. Examples in the nutrition domain include the protein catabolic rate, typically expressed in g/day, and the normalized protein catabolic rate, typically expressed in g/kg of body weight/day, as well as the serum phosphorus level. Examples in the inflammatory domain include the white and red blood cell counts, and indices derived from them, such as, for example, the neutrophil to lymphocyte ratio. Examples in the anthropometric domain include body mass index and body composition indices.

An “alert” level, notifying a physician that a patient is at increased risk of death, can be established by detecting a substantial change in the rate of decline or the rate of increase (e.g., for white blood cell count and neutrophil/lymphocyte ratio) of at least one of the clinical and biochemical parameters discussed above, or any combinations of them. The substantial change that triggers a physician notification is, of course, a substantial change in the same direction, that is, a substantial increase in the rate of increase, or a substantial decline in the rate of decline.

When a patient is “alert” flagged, certain diagnostic procedures can be triggered. These includes, but are not limited to 1) the taking of a thorough history and physical examination with the specific aim to search for cardiovascular, inflammatory, and infectious conditions, 2) blood tests, including C-reactive protein (CRP), albumin, red and white cell blood counts, troponin, blood cultures, 3) echocardiogram, electrocardiogram, 4) Chest x-ray, 5) imaging, in particular ultrasound, computer tomography and/or magnetic resonance imaging, 6) endoscopy, and 7) bacterial cultures and swabs.

Three broad categories of diagnoses can account for >80% of all diagnoses: cardiovascular disease (especially congestive heart failure (CHF) and coronary artery disease (CAD)); inflammation; and infection.

In cases of CHF and/or CAD, therapeutic interventions include but are not limited to strict volume control, which includes avoidance of intradialytic administration of sodium and sodium loading via the dialysate, dietary sodium intake below 6 g/day, increased dialysis frequency, drug therapy (angiotensin converting enzyme inhibitors [ACEI], angiotensin receptor blockers [ARB]1 beta blockers [BB]), lipid lowering drugs, replacement of deficient hormones, valve repair, and percutaneous transluminal coronary angioplasty.

In cases of inflammation without evidence of infection, therapeutic interventions include but are not limited to removal of in-dwelling lines and catheters, therapy with anti-inflammatory drugs, broad spectrum antibiotic therapy, treatment of periodontal disease, and removal of rejected transplants and non-functioning vascular access.

In cases of infection, therapeutic interventions include but are not limited to antibiotic therapy, mechanical and chemical debridement, and removal of in-dwelling lines and catheters.

In all “alert” flagged patients a comprehensive nutritional assessment is usually warranted. In cases of poor nutritional status, therapeutic interventions can include but are not limited to intradialytic parenteral nutrition and oral supplements.

All of the previously described diagnostic and therapeutic interventions on patients are more effective with earlier identification that the hemodialysis patient is at an increased risk of death, with 12 weeks or 3 months of lead time being sufficiently early for an effective intervention.

The relevant teachings of all patents, published applications and references cited herein are incorporated by reference in their entirety.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of identifying a patient undergoing periodic hemodialysis treatments at increased risk for death, comprising:
a) determining at least one of the patient's systolic blood pressure, serum albumin level, body weight, and body temperature periodically while the patient is undergoing hemodialysis treatments; and
b) identifying the patient as having an increased risk for death if the patient has a substantial change in the rate of decline of at least one of the patient's systolic blood pressure, serum albumin level, body weight, and body temperature.

2. The method of claim 1 wherein identifying the patient as having an increased risk of death is determined because the patient has a substantial change in the rate of decline of systolic blood pressure, serum albumin level, body weight, and body temperature.

3. The method of claim 1 wherein identifying the patient as having an increased risk of death is accomplished within a sufficient lead time to allow for a therapeutic intervention to decrease the patient's risk of death.

4. A method of identifying an increased mortality risk factor for a patient undergoing periodic hemodialysis treatment, comprising:
a) analyzing data in deceased patients that were previously undergoing periodic hemodialysis treatments by performing a longitudinal analysis backwards in time of changes in a clinical or biochemical parameter of the patients; and
b) identifying a substantial change in the rate of decline or the rate of increase of a clinical or biochemical parameter before death of the patients.
